(19) United States
(12) Patent Application Publication
(10) Pub. No.: US 2025/0366922 A1
(43) Pub. Date: Dec. 4, 2025

(54) SEMI-RADIOLUCENT PATIENT-SPECIFIC 3D-PRINTED LATTICE TITANIUM PLATE

(71) Applicant: NEW YORK UNIVERSITY, New York, NY (US)

(72) Inventors: Jamie P. Levine, Scarsdale, NY (US); Kenneth S. Hu, New York, NY (US); David A. Daar, New York, NY (US); David J. Byun, New York, NY (US); Martha Malin, New York, NY (US); Lavinia Anzai, New York, NY (US); Adam S. Jacobson, Tenafly, NJ (US); David Barbee, Long Island City, NY (US); Lukasz Witek, Jersey City, NJ (US)

(73) Assignee: NEW YORK UNIVERSITY, New York, NY (US)

(21) Appl. No.: 18/877,884

(22) PCT Filed: Jun. 22, 2023
(86) PCT No.: PCT/US2023/026014

§ 371 Date: Dec. 20, 2024

Related U.S. Application Data
(60) Provisional application No. 63/354,780, filed on Jun. 23, 2022.

(51) Int. Cl. A61B 34/10; A61B 17/00; A61B 17/56; A61B 17/80; A61L 27/06; A61L 27/34; B33Y 40/20; B33Y 50/00; C22C 14/00

(52) U.S. Cl. CPC A61B 34/10; A61B 17/8071; A61L 27/06; A61L 27/34; B33Y 40/20; B33Y 50/00; C22C 14/00; A61B 2017/00526; A61B 2017/0092; A61B 2017/568; A61B 2034/108; A61L 2430/02

(57) ABSTRACT

A plate for fixating bone, the plate comprising: a plurality of ribs defining a plurality of openings, wherein a first portion of the plurality of ribs define a lattice structure and a second portion of the plurality of ribs define at least four mounting holes. Another aspect of the present disclosure relates to a method for tailoring a plate for fixating bone. The method can include determining an initial design of the plate. The method can include analyzing radiation through the initial design. The method can include determining, based on analyzed radiation, if dosimetric characteristics are desirable. The method can include analyzing structural strength of the initial design. The method can include determining, based on the analyzed structural strength, if structural strength is sufficient.

### CROSS-REFERENCE TO RELATED APPLICATIONS

The present application claims the benefit of priority to U.S. Provisional Application No. 63/354,780, filed Jun. 23, 2022, the entire contents of which are incorporated herein by reference.

### TECHNICAL FIELD

The present disclosure relates generally to plates for mitigating bone loss from mandibular resection.

### BACKGROUND

Treatment of various types of conditions can include the removal of tissue (e.g., hard and soft) and replacement with a biocompatible replacement. For example, cancer treatments may involve the removal of cancerous tissue as well as additional surrounding noncancerous tissue (e.g., margins). This tissue may include hard tissue such as bone. Further, tissue necrosis, particularly bone necrosis may occur as the result of radiation used to treat a cancer.

### SUMMARY

One aspect of the present disclosure relates to a plate for fixating bone. The plate can include a plurality of ribs defining a plurality of openings. A first portion of the plurality of ribs can define a lattice structure and a second portion of the plurality of ribs can define at least four mounting holes.

Another aspect of the present disclosure relates to a method for tailoring a plate for fixating bone. The method can include determining an initial design of the plate. The method can include analyzing radiation through the initial design. The method can include determining, based on analyzed radiation, if dosimetric characteristics are desirable. The method can include analyzing structural strength of the initial design. The method can include determining, based on the analyzed structural strength, if structural strength is sufficient. The method can include altering, responsive to determining at least one of the dosimetric characteristics being undesirable or the structural strength being insufficient, the initial design to result in an altered design. The method can include fabricating, responsive to determining that the dosimetric characteristics are desirable and the structural strength is sufficient, the plate.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. The foregoing information and the following detailed description and drawings include illustrative examples and should not be considered as limiting.

### BRIEF DESCRIPTION OF THE FIGURES

The disclosure will become more fully understood from the following detailed description, taken in conjunction with the accompanying figures, wherein like reference numerals refer to like elements, in which:

FIG. 1 illustrates a perspective view of a plate, according to an example implementation.

FIG. 2 illustrates a front view of the plate of FIG. 1, according to an example implementation.

FIG. 3 illustrates a top view of the plate of FIG. 1, according to an example implementation.

FIG. 4 illustrates a side view of the plate of FIG. 1, according to an example implementation.

FIG. 5 illustrates front view of a plate, according to an example implementation.

FIG. 6 illustrates a diagram of radiation passing through a plate, according to an example implementation.

FIG. 7 illustrates as flow diagram of a method of tailoring a plate, according to an example implementation.

FIG. 8 illustrates a side view of a plate affixed to a mandible, according to an example implementation.

FIGS. 9A and 9B illustrate stress/strain graphs of plates, according to an example implementation.

FIGS. 10-12 illustrate perspective views of a plate, according to an example implementation.

FIGS. 13 and 14 illustrate perspective views of a plate affixed to a mandible, according to an example implementation.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting. Examples of specific implementations and applications are provided primarily for illustrative purposes.

The present disclosure is directed to system, methods, and apparatuses related to the design and fabrication of a patient-specific (e.g., designed/optimized for each patient) plate configured to fixate mandibular bone to autologous (e.g., obtained from same individual) bone within a defect and/or bone gap resulting from oncologic (e.g., cancer-related) resection, orthopedic surgery, radiology, trauma, neurosurgery, dentistry, oral maxillofacial surgery, thoracic surgery, or the like. In some embodiments, the plate also reduces artifacts during diagnostic imaging, when compared to traditional plates, thus facilitating better surveillance (e.g., monitoring of disease). The plates may also be used in various locations throughout the body. In some embodiments, the plate is designed iteratively to determine a design that optimizes for radiolucency (e.g., radiation pass-through), while maintaining a strength threshold for mandibular bite-force. In some embodiments, the plate is manufactured using a 3-dimensional (3D) printing method. In some embodiments, the plate is made of a biocompatible titanium alloy having as lattice structure within a central portion composed of narrow supportive beams. In some embodiments, the beams are arranged into parallelogram and/or triangular shapes and are interspersed with holes used as mounting points for mounting the plate to the mandibular bone. In some embodiments, the plate is bent or conformed preoperatively based on a 3D mandibular model of the patient.

As used herein, the term “mandibular” refers to the largest bone of the jaw, the mandible. The mandible houses the teeth and is a key component of mastication (e.g., chewing), and must thus be structured (with or without support) to support the forces associated with mastication.

As used herein, the term “radiation” in reference to therapeutic methods and treatment refers to any type of therapy using localized ionizing radiation to control or kill cancer cells, such as external beam photon therapies (intensity modulated radiation therapy (IMRT), 3D conformal radiation therapy (3DCRT), image guided radiation therapy (IGRT), volumetric modulated arc therapy (VMAT), or the like), external beam particle therapies (e.g., electrons, protons, heavy charged particles, etc.), brachytherapy isotopes and treatments, or targeted/systemic radionuclides. Certain materials and designs may excessively attenuate radiation by unknown or varying amounts or may result in increased radiation scatter that may be unaccounted for in dose calculation algorithms and lead to inaccuracies in dose distribution calculations in radiation treatment.

The removal of bone tissue, whether as part of a tumor resection or resulting from or in avoidance of bone necrosis due to radiation, can be accompanied by the use of a plate such as a titanium plate for fixation. Computerized surgical planning (CSP) and other methods such as Virtual Surgical Planning (VSP) can be used to design a course of treatment specific to a patient as well as to aid in the design of a plate specific to the patient's condition and anatomy.

In cases of both head and neck cancer, bone necrosis may occur at the site of radiation. Radical mandibular resection (e.g., removal of all or part of the jaw bone) may be necessary to remove sections of the bone with necrosis. To reconstruct the mandible, osseous reconstruction fixated to the native (e.g., original) mandible with a single, locking titanium reconstruction plate can be used.

In many cases, radiation therapy may still be needed, even after mandibular resection. CSP can typically be designed independent of the planned radiation treatment. Traditional reconstruction plate designs pose a major difficulty to radiation therapy planning and delivery, as backscatter from the reconstruction plate may not be modeled by dose calculation algorithms, leading to problems associated with osteoradionecrosis, local recurrence, and reconstructive failure. These issues may lead to worsening pain, oral function, and overall quality of life for the patient.

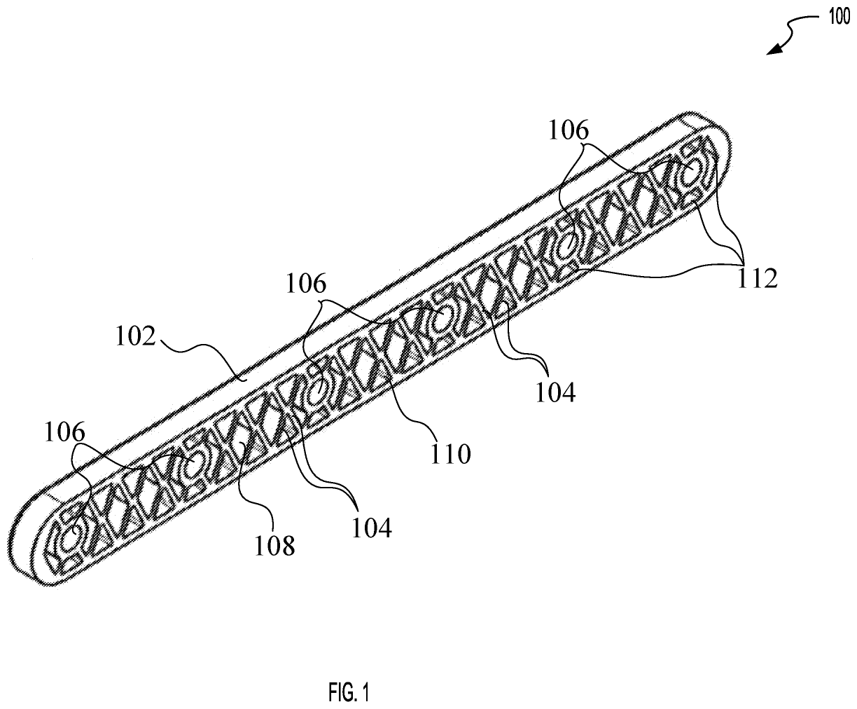

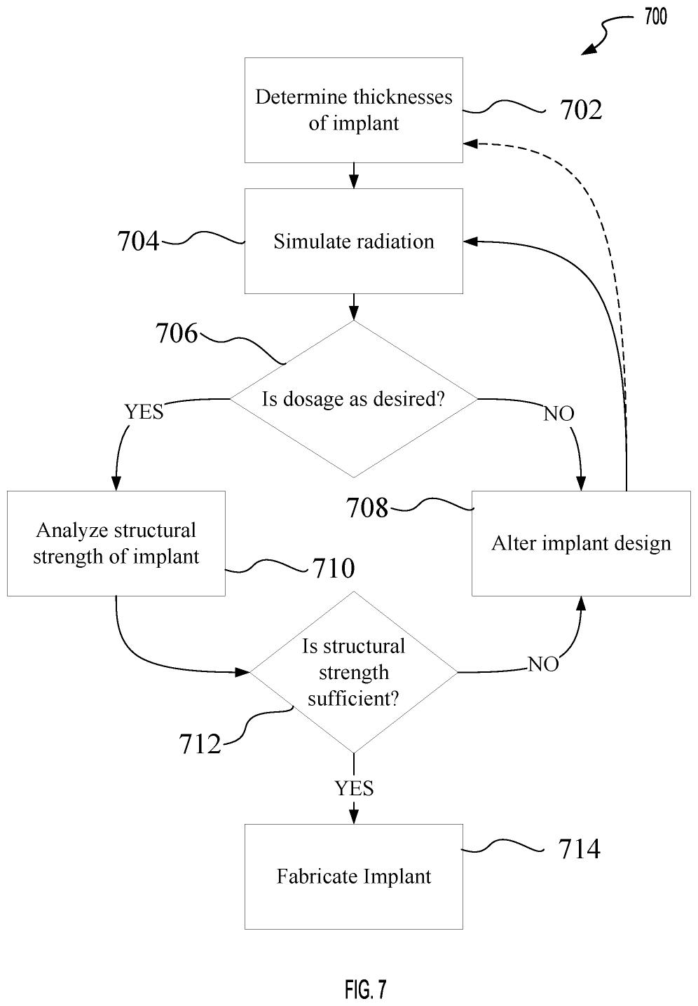

Referring generally to FIGS. 1-5, several plates are shown. The plates in the aforementioned figures all appear planar (e.g., flat, having a face along a plane, etc.) as they may appear, in some embodiments, after an initial manufacturing step (e.g., casting, 3D printing, etc.). The plates may be bent (e.g., reconfigured, twisted, molded, shaped, etc.) to correspond to the exact shape of a patient's anatomy, such as a mandible. Furthermore, the plates of the aforementioned figures appear symmetric both vertically and horizontally. However, this is not limiting, as plates may include designs that are non-symmetric (e.g., non-symmetric hole distribution/rib configuration/etc.). A further discussion of design consideration and fabrication is discussed in reference to FIG. 7.

In some embodiments, the plates of FIGS. 1-5 are formed of a bio-compatible material, such as a titanium alloy (e.g., Ti-6Al-4V). The plates can be formed of titanium. In some embodiments, the plates are formed in a unitary construction or may be composed of several components fixedly coupled (e.g., welded, screwed, adhered, etc.). The design may be configured to fix to remaining bone structure of a specific patient and include a “pre-bent” (that is before implantation) design modeled based on the patient's anatomy. The design of the plate may be selected to reduce the interference with radiation, such as by utilizing a minimally radiopaque design to reduce the interference by the plate. In some embodiments, several components may include selective coupling devices (e.g., hinges, etc.) to adjust the plate. In some embodiments, the plates may include a coating, such as an antibacterial coating, anticorrosive coating, or the like. In some embodiments, the plates may include a radiolucent biocompatible membrane. In some embodiments, the radiolucent biocompatible membrane is resorbable. The membrane may include a thin biomaterial. For example, the thin biomaterial can includes a porcine pericardium collagen that is wrapped around the plates. The membrane can maintain a microporous tissue architecture (e.g., multilayered, 3-layered, etc.) that substantially resorbs, effectively creating a barrier to tissue ingrowth. The membrane may be placed to hinder soft tissue migration into the lattice structure and may allow for ease of plate manipulation and/or removal in the event that revision surgery in necessary. In some embodiments, the plates may be scaled up or down.

Referring now to FIG. 1, a perspective view of a plate 100 (e.g., implant) is shown. The plate 100 is configured to fixate mandibular bone to autologous bone within a defect/gap in a patient. The plate 100 supports the mandible during osseous (e.g., relating to bone) reconstruction and/or bony healing and provides biomechanical structural support during biomechanical movements such as mastication. The benefits of the plate 100 can improve the quality of life of a patient, while minimizing negative impacts on future radiation therapy.

The overall shape of the plate 100 can be defined by an outside edge 102. In the embodiment of FIG. 1, the outside edge 102 can include two parallel portions capped by semi-circular portions. In some embodiments, the outside edge 102 may define another regular (e.g., symmetric, etc.) or irregular (e.g., non-symmetric, including multiple shapes, etc.) shape. The outside edge 102 can be continuous (e.g., forms a closed loop). The outside edge 102 can serve as a frame for the plate 100. The outside edge 102 can surround a plurality of ribs 104 (e.g., trusses, supports, braces, etc.) disposed within plate 100. The ribs 104 can provide the plate 100 with structural support. For example, the ribs 104 may distribute biomechanical forces (e.g., forces associated with biomechanical movements) throughout the plate 100 to minimize deflection and increase rigidity. The ribs 104 may be linear (e.g., straight), or may include at least one curve. The ribs 104 may define a pattern (e.g., lattice, weave, etc.) along at least one characteristic measurement (e.g., length, width, etc.) or may be designed specifically for the application of the plate 100. The plurality of ribs 104 can allow for radiolucency. The plurality of ribs 104 can define a lattice structure. For example, the plurality of ribs 104 can be configured in the lattice structure. The lattice structure can include at least one of triangles, diamonds, or parallelograms.

The plurality of ribs 104 can define a plurality of openings. The openings can pass through the width of the plate 100. The openings can include a plurality of mounting holes 106. The plate 100 may have any number of mounting holes (e.g., 1, 2, 3, 4, 5, 6, 7, 8, 9, 10, 15, 20, 25, etc.) The mounting holes 106 can be configured to receive a fastener (e.g., screw, bolt, etc.) for coupling the plate 100 to the bone of a patient. The location of the mounting holes 106 may correspond to the locations of bone in the patient capable of receiving a fastener. In some embodiments, the mounting holes 106 may be arranged symmetrically along the plate 100. In some embodiments, the plate 100 may not include a mounting hole 106 and may be coupled to the bone via an adhesive (e.g., glue, epoxy, etc.). The mounting holes 106 may be threaded to couple with a screw or may be smooth to allow for a bolt to pass through and/or rotate. In some embodiments, the mounting holes 106 may include a specific shape corresponding to a through bolt to prevent rotation. For example, if a through bolt is hexagonal, the mounting holes 106 may be hexagonal. In some embodiments, the mounting holes 106 may vary in the plate 100. For example, a subset of the mounting holes 106 may be hexagonal, while another subset may be circular. As another example, the mounting holes 106 may all be circular, but have different radii. It should be appreciated that the mounting holes 106 may be specifically positioned with regard to a particular patient's anatomy and needs.

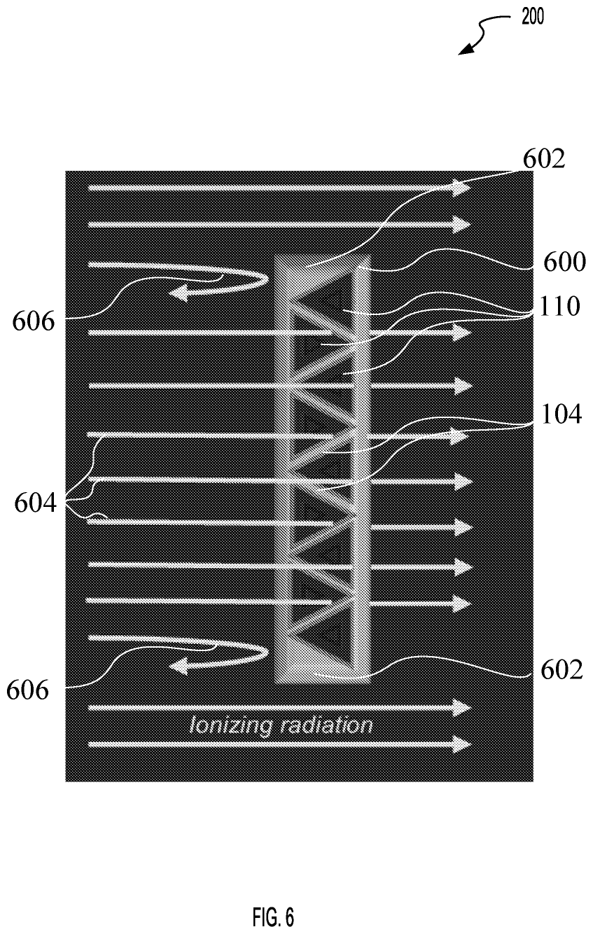

The openings can also include various openings designed to allow for radiation to pass through (further discussed in reference to FIG. 6). The shape of the openings can be configured to increase the compressive and tensile strength of the plate 100. In some embodiments, the openings may define a truss shape (e.g., structure connected by nodes). The openings can include parallelogram openings 108 and/or triangular openings 110. The parallelogram openings 108 and/or the triangular openings 110 may be arranged in a pattern to evenly distribute biomechanical forces along the length of the plate 100. The plate 100 also includes a plurality of irregular openings 112. The irregular openings 112 may be added for additional weight savings, or surrounding other openings such as, in FIG. 1, the mounting holes 106.

The layout of the openings may affect the structural strength of the plate 100 and deflections when experiencing biomechanical forces. The layout of the openings can also affect how much and where radiation may pass through. The plate 100 may be specifically designed to be stronger in certain areas and/or allow for additional radiation to pass through or shield in certain areas.

The plurality of ribs 104 can be encapsulated by a membrane. For example, a membrane can encapsulate the plurality of ribs 104. The membrane can include collagen. Collagen can encapsulate the plurality of ribs 104. The first portion of the plurality of ribs 104 can be inlayed with a membrane. For example, the first portion of the plurality of ribs 104 can be inlayed with a radiolucent biocompatible membrane. The second portion of the plurality of ribs 104 can be inlayed with the membrane. For example, the first portion of the plurality of ribs 104 can be inlayed with the radiolucent biocompatible membrane.

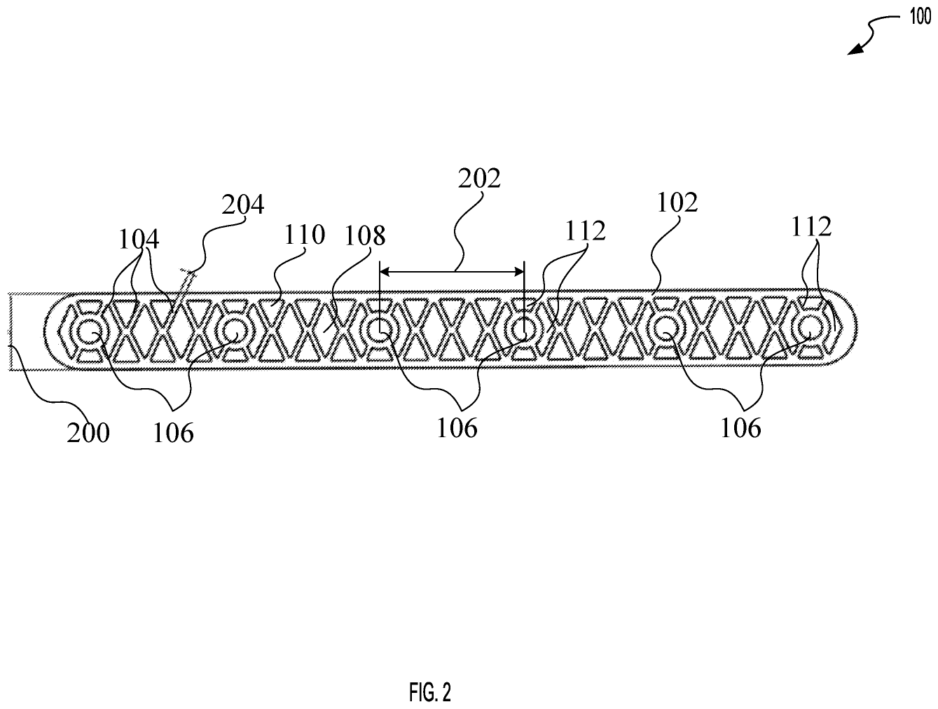

Referring now to FIG. 2, a front view of the plate 100 of FIG. 1 is shown. The plate 100 can include a width 200, which can be defined as the distance between the outside of the two parallel portions of the outside edge 102. The width 200 can be configured specifically for the patient. For example, the width 200 may be smaller when designed for a child than when designed for an adult. The plate 100 can include openings arranged in a pattern. The pattern, along the length of the plate 100, can include mounting holes 106 surrounded by irregular openings 112, then three sets of stacked triangular openings 110 with parallelogram openings interposed, before another mounting hole 106 surrounded by irregular openings. The irregular openings 112 may be continuations of the opening patterns, but with the mounting holes 106 filling in portions. In some embodiments, the plate 100 may include varying patterns, wherein certain patters may be configured or optimized for strength, radiolucency, etc. The plate 100 of FIG. 1 includes six total mounting holes 106. The first hole distance 202, which can be defined by the distance between the mounting holes 106, may be constant between the mounting holes 106 or may vary between pairs of mounting holes 106. The first hole distance 202 may vary based on the portions of the bone of the patient that are capable of accepting a fastener. The mounting hole 106 pattern of the plate can include even spacing.

FIG. 2 also shows the plurality of ribs 104 defining a rib width 204, which can be defined by the width of each or all of the plurality of ribs 104. The rib width 204 may be constant along the length of each rib of the plurality of ribs 104, or may vary. For example, the rib width 204 may increase near the point where one rib of the plurality of ribs 104 couples to (e.g., contacts) another rib of the plurality of ribs 104. The rib width 204 may be constant from rib to rib rib of the plurality of ribs 104, or may be varied from rib to rib rib of the plurality of ribs 104. The plurality of ribs 104 in areas undergoing higher biomechanical forces may be thicker to provide additional structural support. For example, the plurality of ribs 104 around the mounting holes 106 may be thicker as the mounting holes 106 may experience higher biomechanical forces. The plurality of ribs 104 may be thinner in areas with may need localized radiation. The plate 100 may have a minimal feasible rib width, which corresponds to minimum rib width that can feasibly be manufactured.

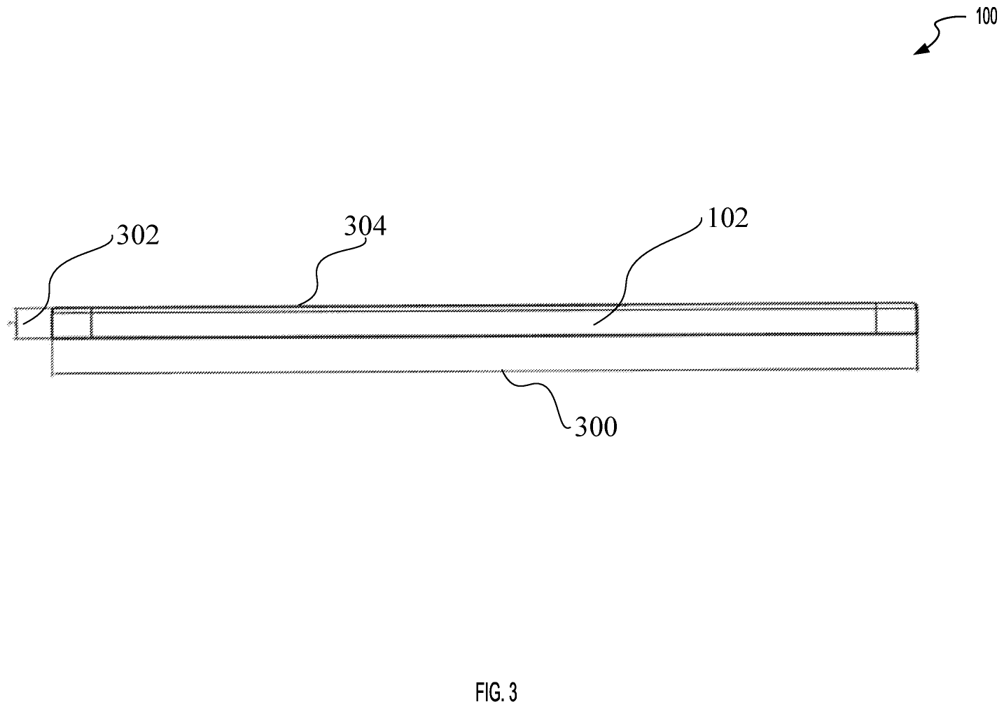

Referring now to FIG. 3, a top view of the plate 100 of FIG. 1 is shown. The plate 100 can include a length 300. The length 300 can be defined as the longest characteristic measurement of the plate 100. The length 300 is configured specifically for the patient. For example, the length 300 may be smaller when designed for a child than when designed for an adult. The plate 100 also includes a thickness 302. The thickness 302 can be defined as the width of the outside edge 102. In some embodiments, the length 300 is larger than the width 200, which is larger than the thickness 302. The plate 100 includes a chamfer 304 located along an edge of the outside edge 102. The chamfer 304 may decrease the possibility of stress concentrations within the plate 100.

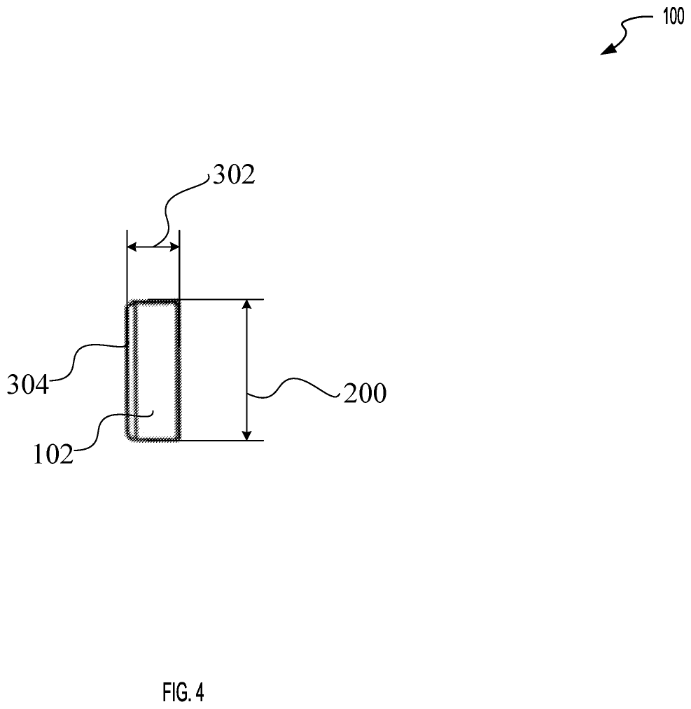

Referring now to FIG. 4, a side view of the plate 100 of FIG. 1 is shown. As seen in FIG. 4, the chamfer 304 may be along the entirety of an edge of the outside edge 102. The side profile of the plate 100 is approximately rectangular. In some embodiments, both edges of the outside edge 102 may be chamfered.

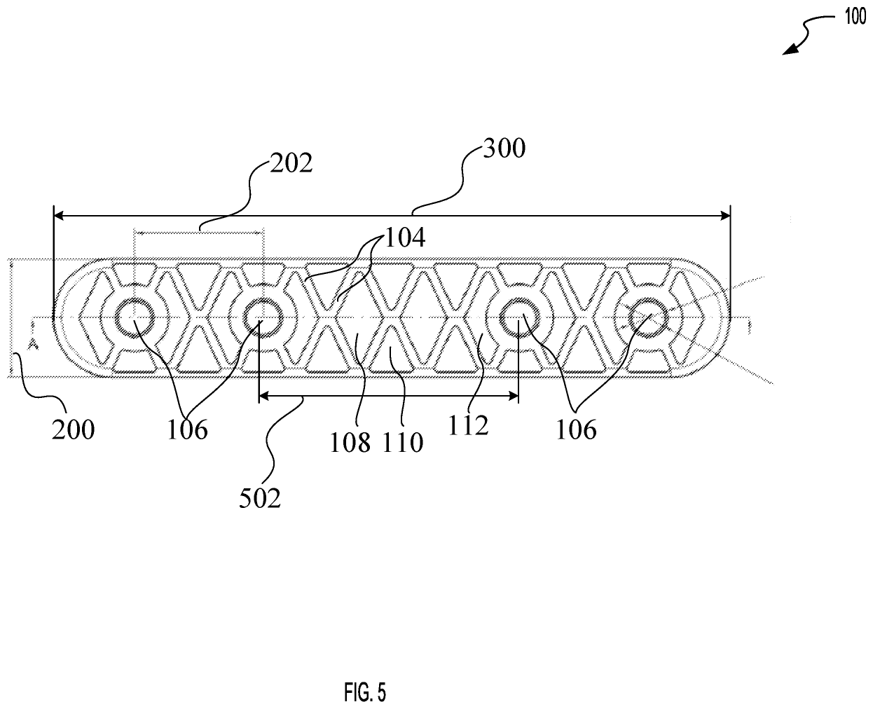

Referring now to FIG. 5, a front view of the plate 100 is shown. The plate 100 can feature a pattern of repeating parallelogram openings 108 interposed between stacked triangular openings 110. The plate 100 can include four mounting holes 106. The mounting holes 106 of the plate 100 can have a variable spacing, with one pair having the first hole distance 202, a next pair having a second hole distance 502, and another pair having the hole distance. Irregular openings 112 can surround the mounting holes 106. The width 200 and the length 300 of the plate 100 may be the same or different than the width 200 and the length 300 of the plate 100 shown in FIG. 1. If the plate 100 of FIG. 5 has the same width 200 and the same thickness 302 as, but a smaller length 300 than the plate 100 shown in FIG. 1, the plate 100 of FIG. 5 may be used with patients having a resection.

Additional embodiments of plates may include different configurations of openings, thickness 302, width 200, lengths 300, mounting holes 106, shapes, patterns, etc. to meet the needs of a patient and structural and radiolucency goals. In some embodiments, a collection of designs varying by configuration may be automatically generated allowing the operator to select a design from the collection.

Referring now to FIG. 6, a diagram of radiation passing through an exemplary plate 600 is shown. The diagram illustrates the effects of including openings in a plate on ionizing radiation. FIG. 6 includes the exemplary plate 600 as a stand-in for a plate, such as the plate 100. The exemplary plate 600 can include a plurality of triangular openings 110 defined by a plurality of ribs 104. The exemplary plate can include one or more reinforced portions 602, which include more material than the exemplary plate 600 includes at the plurality of ribs 104. In FIG. 6, ionizing radiation can be directed at the exemplary plate 600. Rays of the ionizing radiation directed towards the triangular openings 110 can pass through the exemplary plate 600 (e.g., passing rays 604), while the rays directed at the reinforced portion 602 can be deflected (e.g., deflected rays 606). The deflected rays may deflect back towards the ionizing radiation source or may be misdirected, both decreasing the delivered dose behind the plate and increasing the dose at the entrance interface of the plate. Thus the openings of the plates can be configured to reduce radiation attenuation, and thus decrease scatter dose enhancement, allowing for a relatively greater fraction of radiation to pass through and reach a target location for delivering doses more accurately than when using traditional plates.

Referring now to FIG. 7, a flow diagram of a method 700 of tailoring the plate 100 is shown. The steps of method 700 may be completed, partially completed, or facilitated by a computing system. The computing system can include at least a processor and a memory storage. The memory storage may include machine-readable instructions that may be executed by the processor. The computing system may include a communications interface configured to communicate with other computing system or with hardware (e.g., manufacturing hardware, communication hardware, input/output device, etc.). In some embodiments, the computing system may be operating in parallel and processing at least two of the steps of the method 700 simultaneously. The method 700 beings with an initial design for a plate already determined based on a plurality of set design points, such as mounting hole 106 locations. The initial design may be a predefined “standard” design or a design chosen by the physician as best suited for the patient. This initial design is then iterated through and optimized to meet patient and physician needs for both treatment and quality of life improvements for the patient.

At step 702, the thickness (e.g., thickness 302) of the plate can be determined. The method 700 can include determining an initial design of the plate. The determination may be based on the amount of space available along the mandible of the patient, the side of the autologous bone, radiation planning, patient needs, structural consideration, anatomic location for reconstruction, or other similar design points. In some embodiments, the thickness may be predetermined or predefined by a physician or based on historical data. The plate can be formed of a biocompatible material. The biocompatible material can include Ti-6Al-4V titanium alloy. The plate can be formed of titanium. The plate can include a plurality of ribs defining a lattice structure.

At step 704, radiation can be simulated through the plate (e.g., an initial design of the plate). The method 700 can include analyzing radiation through the initial design. The simulation may rely on radiation dose calculation using Monte Carlo to assess areas of potential dose enhancement or shadowing to surrounding tissue and bone. Dose calculation can be performed at multiple angles relative to surface normal of the plate design to identify potential combinations of radiation beam trajectories and to identify plate design that result in unacceptable dosimetric properties. The simulation results may then be analyzed using dosimetric analysis to determine areas that may have insufficient results, as determined by a physician, physicist, look-up table, algorithm, etc. Simulating and analyzing radiation through the plate may highlight areas that may need to be reduced to custom-fit the plate to the treatment plan. Information from radiation simulation may be used to inform subsequent iterations of plate design. Analyzing the radiation through the initial design can include analyzing radiation through the initial design via a Monte Carlo simulation.

At step 706, a determination can be made whether the dosimetric characteristics from the simulated radiation is within desired ranges and is acceptable for treatment. The method 700 can include determining, based on analyzed radiation, if dosimetric characteristics are desirable (e.g., undesired). If the dosimetric characteristics of the proposed patient specific plate are found to be at an undesired level, whether too high or too low, the plate design can be altered at 708. At step 708, the configuration (e.g., shape, openings, opening density, rib width 204, etc.) may be altered to achieve the desired dosimetric properties. Alterations may be done manually (e.g., by a physician, designer, etc.) or automatically by the computing system. In some embodiments, the alterations may be responsive to the simulation highlighting areas outside acceptable ranges, which are then altered. All dosimetric simulations may be saved such that alterations may be compared to radiation simulations of previous designs. In cases where multiple possible designs are generated with radiation simulations, cases may be intercompared for the user to select the ideal plan. The alterations may be limited by a minimal rib width, a minimal opening density, or similar parameter. In some embodiments, after initial alterations are completed, the thickness may be redesigned. After all alterations are complete, the method 700 returns to step 704 to again simulate radiation through the plate. Step 704 (and sometimes step 702), step 706, and step 708 may be repeated until the dosimetric characteristics of the plate are determined to be as desired, after which the method 700 proceeds to step 710.

At step 710, the structural strength of the plate can be analyzed. The method 700 can include analyzing the structural strength of the initial design. In some embodiments, finite element analysis (FEA) is used to determine the amount of radiopaque plate material interfacing with bone is necessary to maintain a strength range. In some embodiments, the strength range may correspond to an average mandibular bite force in a post-reconstruction mandible. In some embodiments the strength range may be approximately 148 Newton (N) to 187 N for a mandibular bite force at a first molar during mastication. Analyzing the structural strength of the initial design can include analyzing the structural strength of the initial design via finite element analysis.

At 712, a determination can be made whether the structural strength is sufficient (e.g., within the strength range). The method 700 can include determining, based on the analyzed structural strength, if structural strength is sufficient. If the determination is that the structural strength is insufficient (e.g., outside of the strength range), the method 700 can return to step 708. The method 700 can include altering, responsive to determining at least one of the dosimetric characteristics being undesirable or the structural strength being insufficient, the initial design to result in an altered design. The initial design can include a first amount of material and the altered design can include a second amount of material. The second amount of material can be less than the first amount of material. The second amount of material can be equal to the first amount of material. The second amount of material can be greater than the first amount of material. The first amount of material can be less than the second amount of material. The first amount of material can be greater than the second amount of material.

At step 708, the plate design can be altered agin, with an emphasis on structural strength. For example, if the structural strength was found to be above a desired range, step 708 may remove a rib of the plurality of ribs 104 from the plate to reduce the structural strength and increase radiolucency. The desired range for structural strength may be determined as the strength desired so that the healing bone and/or autologous bone may encounter sufficient stress for healing. Structural strength above a desired range may cause a stress-shielding effect for the healing bone and/or autologous bone which may cause the bone to resorb and decreases the likelihood of a bony union. The removed rib of the plurality of ribs 104 may correspond to a rib that the structural analysis indicated carried the lowest forces. Conversely, if the structural strength is found to be too low, step 708 may increase the thickness of certain ribs of the plurality of ribs 104, such as those indicated by the structural analysis as failing and/or experiencing high tensile or compressive forces. In some embodiments, past designs may be “blacklisted” (e.g., disallowed, removed, etc.) such that step 708 does not result in plate designs that have already proved to either result in undesirable dosimetric properties or insufficient structural strength. The method then proceeds to step 704 (or step 702, in some embodiments) to simulate and analyze the dosimetric properties.

If is it determined, at step 712 that the structural strength is sufficient, the method 700 can proceed to step 714. The method 700 can include fabricating, responsive to determining that the dosimetric characteristics are desirable and the structural strength is sufficient, the plate. At step 714, the plate can be fabricated based on the design that the method 700 deemed having desirable dosimetric properties and a sufficient structural strength. The plate may be manufactured out of the material that was used during simulations of step 710 and step 704, as the results of the simulations are dependent on material type. In some embodiments, this material is a titanium alloy, more specifically Ti-6Al-4V titanium alloy.

The plate may be fabricated by casting (e.g., lost wax casting, sand casting, gravity casting, etc.), assembled and coupled, or printed using additive manufacturing (3D printing) (e.g., powder bed fusion, selective last melting, electron beam melting, direct energy deposition (DED), power DED, wire DED, binder jetting, bound powder extrusion, etc.). Fabricating the plat can include fabricating the plate via a 3D printing method. 3D printing can allow for the creation of precise custom-designed components. After fabrication, the plate may undergo several post-processing techniques, such as wrapping with a membrane (as described above), coating, surface finish, painting, deburring, tapping, etc. In some embodiments, the plate may be reshaped during post-processing to best fit to a customer's body. For example, the plate may undergo bending based on a 3D mandibular model developed from a computer tomographic scan imaging of a patient.

The method 700 can include encapsulating the plurality of ribs with collagen. For example, the method can include encapsulating the plurality of ribs with porcine pericardium collagen. The method 700 can include inlaying the plurality of ribs with a membrane. For example, the method 700 can include inlaying the plurality of ribs with a radiolucent biocompatible membrane.

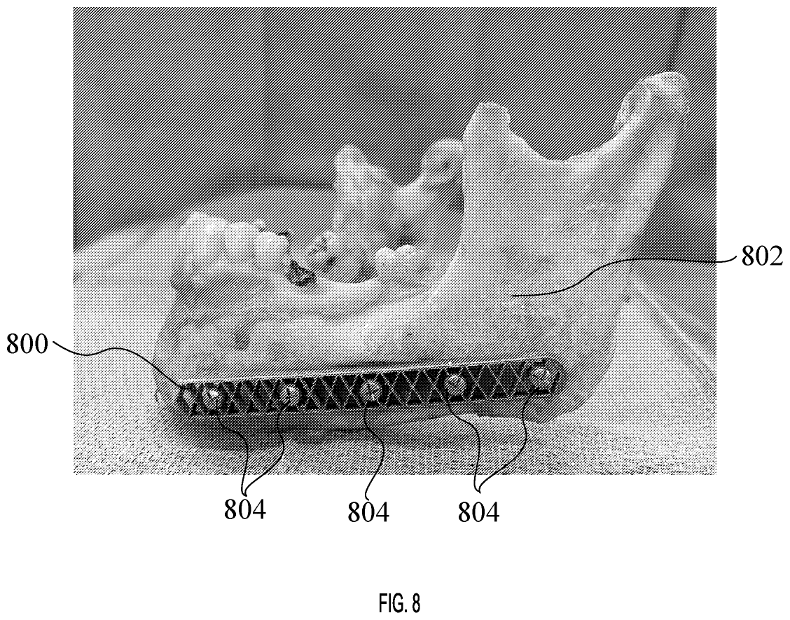

Referring now to FIG. 8, an implant 800, which is similar to the implant 100, affixed to a mandible 802 is shown. The implant 800 can be fastened to the mandible 802 via a plurality of fasteners 804 (e.g., screw, bolt, etc.). The implant 800, as well as the characteristics (e.g., location, size, type, etc.) of the fasteners, can be specifically configured to match the shape of the mandible 802 to provide desirable strength and radiolucency for the patient.

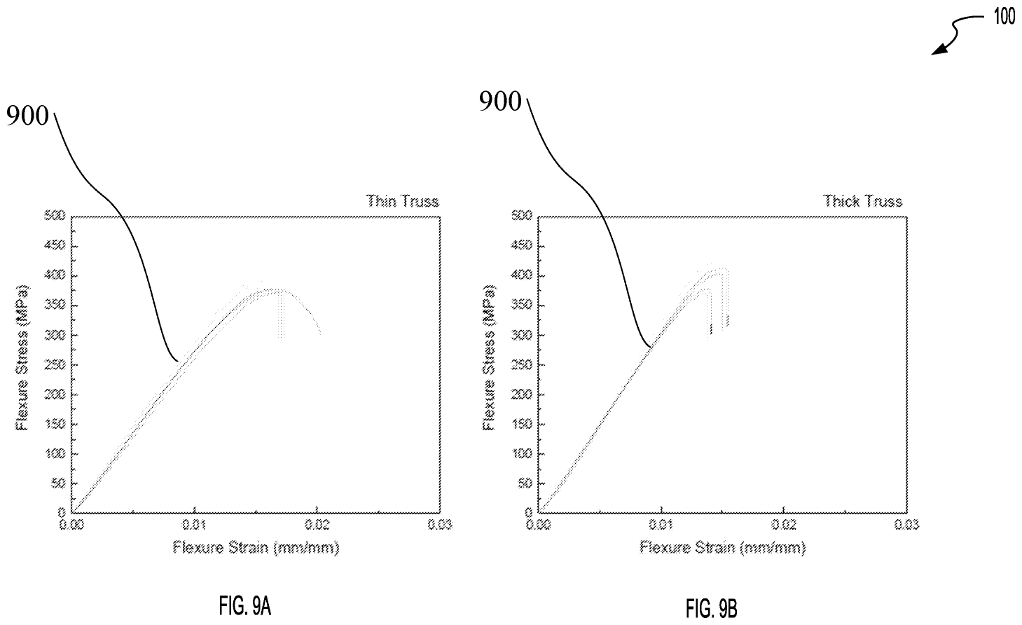

Referring now to FIG. 9A and FIG. 9B, stress/strain graphs of plates are shown. A first plate is shown in FIG. 9A and a second plate is shown in FIG. 9B. The first plate is thinner in width, such as width 200, than the second plate. The stress/strain graphs were determined using 3-point bending flexure test to determine the stress and strain within the first plate and the second plate. The graphs of FIGS. 9A and 9B each include a linear portion 900, which indicates that the first plate and the second plate experience only elastic deformation during a wide range of induced stress.

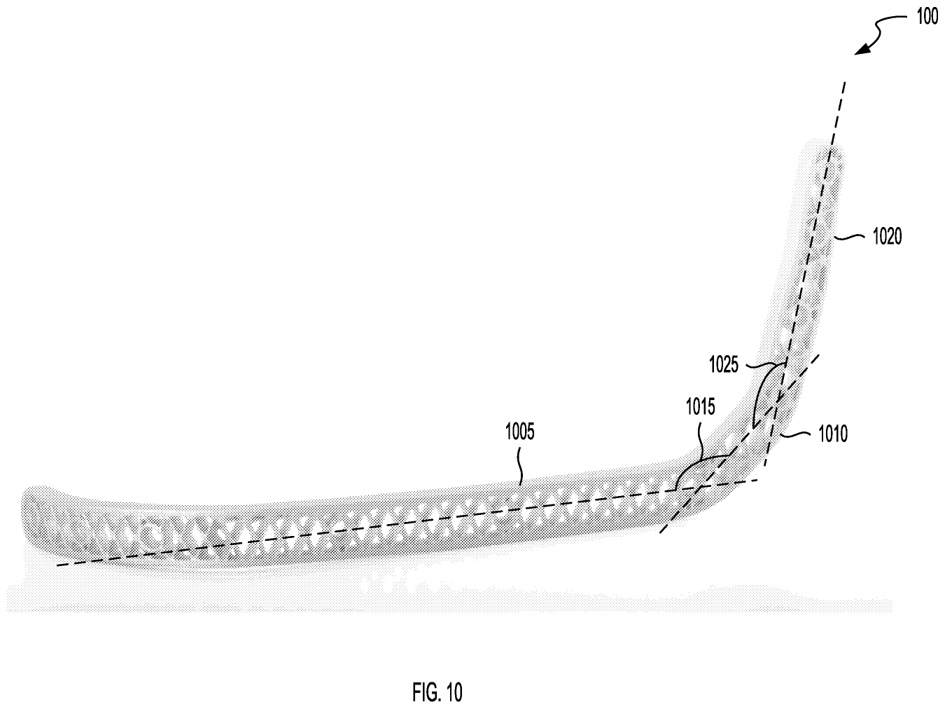

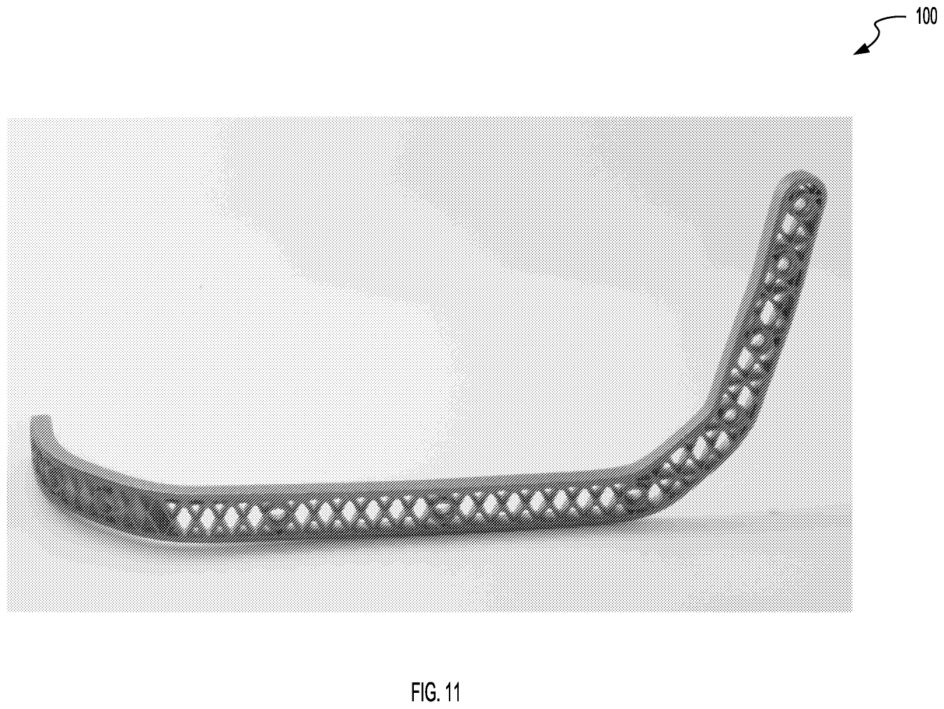

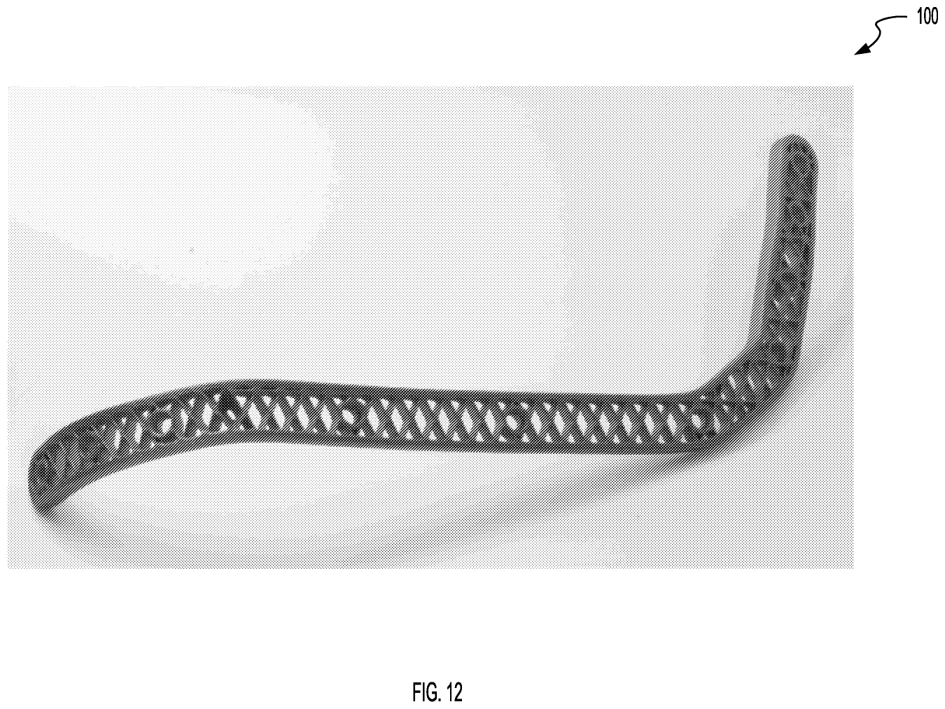

FIGS. 10-12 illustrate perspective views of the plate 100. The plate 100 can be bent. The plate 100 can include a third portion 1005 of the plurality of ribs 104. The plate 100 can include a fourth portion 1010 of the plurality of ribs 104. An angle 1015 between the third portion 1005 of the plurality of ribs 104 and the fourth portion 1010 of the plurality of ribs 104 can be greater than 0 degrees and less than 180 degrees, inclusive. For example, the angle 1015 between the third portion 1005 of the plurality of ribs 104 and the fourth portion 1010 of the plurality of ribs 104 can be 0 degrees, 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, 90 degrees, 95 degrees, 100 degrees, 105 degrees, 110 degrees, 115 degrees, 120 degrees, 125 degrees, 130 degrees, 135 degrees, 140 degrees, 145 degrees, 150 degrees, 155 degrees, 160 degrees, 165 degrees, 170 degrees, 175 degrees, or 180 degrees, inclusive.

The plate 100 can include a fifth portion 1020 of the plurality of ribs 104. An angle 1025 between the fifth portion 1020 of the plurality of ribs 104 and the fourth portion 1010 of the plurality of ribs 104 can be greater than 0 degrees and less than 180 degrees, inclusive. For example, the angle 1025 between the fifth portion 1020 of the plurality of ribs 104 and the fourth portion 1010 of the plurality of ribs 104 can be 0 degrees, 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, 90 degrees, 95 degrees, 100 degrees, 105 degrees, 110 degrees, 115 degrees, 120 degrees, 125 degrees, 130 degrees, 135 degrees, 140 degrees, 145 degrees, 150 degrees, 155 degrees, 160 degrees, 165 degrees, 170 degrees, 175 degrees, or 180 degrees, inclusive.

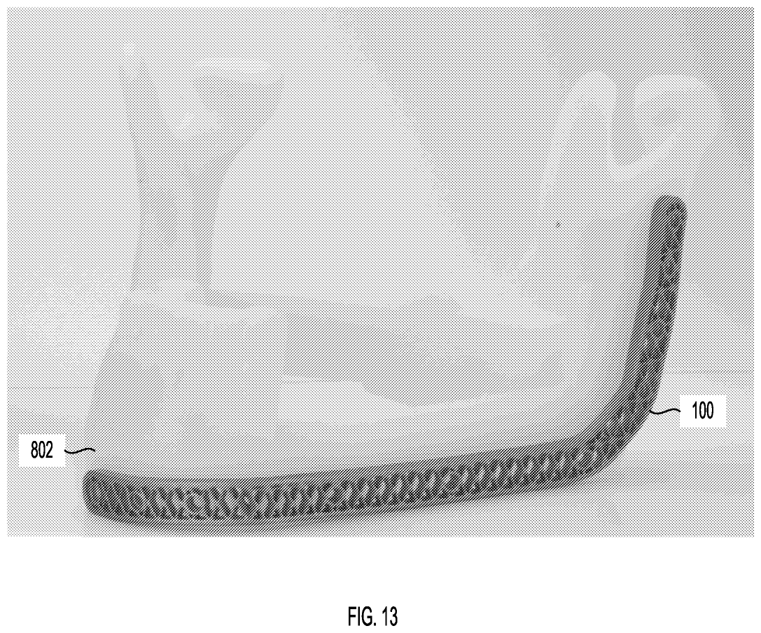

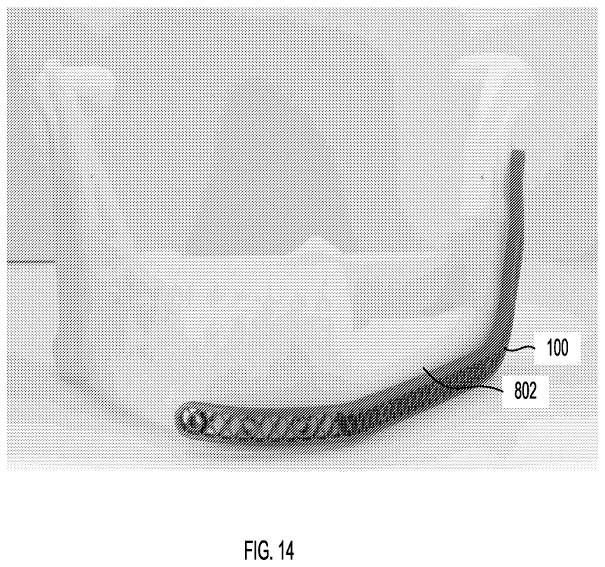

FIGS. 13 and 14 illustrate perspective views of the plate 100. The plate 100 can be affixed to the mandible 802. The plate 100 can be fastened to the mandible 802. The plate 100 can be fastened to the mandible 802 via a plurality of fasteners (e.g., screw, bolt, etc.). The plate 100, as well as the characteristics (e.g., location, size, type, etc.) of the fasteners, can be specifically configured to match the shape of the mandible 802 to provide desirable strength and radiolucency for the patient.

While this specification contains many specific implementation details, these should not be construed as limitations on the scope of what may be claimed but rather as descriptions of features specific to particular implementations. Certain features described in this specification in context of separate implementations can also be implemented in combination in a single implementation. Conversely, various features described in the context of a single implementation can also be implemented in multiple implementations separately or in any suitable subcombination. Moreover, although features may be described as acting in certain combination and even initially claimed as such, one or more features from a claimed combination can, in some cases, be excised from the combination, and the claimed combination may be directed to a subcombination or variation of a subcombination.

As utilized herein with respect to numerical ranges, the terms “approximately,” “about,” “substantially,” and similar terms generally mean ±10% of the disclosed values. When the terms “approximately,” “about,” “substantially,” and similar terms are applied to a structural feature (e.g., to describe its shape, size, orientation, direction, etc.), these terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled direction to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the figures. It should be noted that the orientation of various elements may differ according to other exemplary embodiments, and that such variations are intended to be encompassed by the present disclosure.

Also, the term “or” is used in its inclusive sense (and not in its exclusive sense) so that when used, for example, to connect a list of elements, the term “or” means one, some or all of the elements in the list. Conjunctive language such as the phrase “at least one of X, Y, or Z,” unless specifically stated otherwise, is otherwise understood with the context as used in general to convey that an item, term, etc. may be either X, Y, Z, X and Y, X and Z, Y and Z, or Z, Y, and Z (i.e., any combination of X, Y, and Z). Thus, such conjunctive language is not generally intended to imply that certain embodiments require at least one of X, at least one of Y, and at least one of Z to each be present, unless otherwise indicated.

The hardware and data processing components used to implement the various processes, operations, illustrative logics, logical blocks, modules and circuits described in connection with the embodiments disclosed herein may be implemented or performed with a general purpose single-or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structure and which can be accessed by a general purpose or special purpose computer or other machine with a processor. Combinations of the above are also included in the scope of machine-readable media. Machine-executable instructions include, for example, instructions and data which cause a general purpose computer, special purpose computer, or special purpose processing machines to perform a certain function or group of functions.

It is important to note that the construction and arrangement of the system shown in the various example implementations is illustrative only and not restrictive in character. All changes and modifications that come within the spirit and/or scope of the described implementations are desired to be protected. It should be understood that some features may not be necessary, and implementations lacking the various features may be contemplated as within the scope of the application, the scope being defined by the claims that follow. When the language a “portion” is used, the item can include a portion and/or the entire item unless specifically stated to the contrary.

Although only a few embodiments have been described in detail in this disclosure, those skilled in the art who review this disclosure will readily appreciate that many modifications are possible (e.g., variations in sized, dimensions, structures, shapes, and proportions of the various elements, values of parameters, mounting arrangement, use of materials, colors, orientations, etc.) without materially departing from the novel teachings and advantages of the subject matter described herein. For example, elements shown as integrally formed may be constructed of multiple components of elements, the position of elements may be reversed or otherwise varied, and the nature of number of discrete elements or positions may be altered or varied. The order of sequence of any method processes may be varied or re-sequenced according to alternative embodiments. Other substitutions, modifications, changes, and omissions may also be made in the design, operating conditions and arrangement of the various exemplary embodiments without departing from the scope of the present disclosure.

What is claimed is:

1. A plate for fixating bone, the plate comprising:
a plurality of ribs defining a plurality of openings,
wherein a first portion of the plurality of ribs define a lattice structure and a second portion of the plurality of ribs define at least four mounting holes.

2. The plate of claim 1, wherein the plate is formed of a biocompatible material.

3. The plate of claim 2, wherein the biocompatible material comprises a Ti-6Al-4V titanium alloy.

4. The plate of claim 1, wherein the plate is formed of titanium.

5. The plate of claim 1, wherein the plurality of ribs are configured to allow for radiolucency.

6. The plate of claim 1, wherein the lattice structure comprises at least one of triangles, diamonds, or parallelograms.

7. The plate of claim 1, further comprising:
a membrane configured to encapsulate the plurality of ribs.

8. The plate of claim 1, further comprising:
collagen configured to encapsulate the plurality of ribs.

9. The plate of claim 1, wherein the first portion of the plurality of ribs are inlayed with a radiolucent biocompatible membrane.

10. The plate of claim 1, further comprising:
a third portion of the plurality of ribs; and
a fourth portion of the plurality of ribs;
wherein an angle between the third portion of the plurality of ribs and the fourth portion of the plurality of ribs is greater than 0 degrees and less than 180 degrees.

11. A method for tailoring a plate for fixating bone, the method comprising:
determining an initial design of the plate;
analyzing radiation through the initial design;
determining, based on analyzed radiation, if dosimetric characteristics are desirable;
analyzing structural strength of the initial design;
determining, based on the analyzed structural strength, if structural strength is sufficient;
altering, responsive to determining at least one of the dosimetric characteristics being undesirable or the structural strength being insufficient, the initial design to result in an altered design; and
fabricating, responsive to determining that the dosimetric characteristics are desirable and the structural strength is sufficient, the plate.

12. The method of claim 11, wherein fabricating the plate comprises:
fabricating the plate via a 3D printing method.

13. The method of claim 11, wherein the plate is formed of a biocompatible material.

14. The method of claim 13, wherein the biocompatible material comprises Ti-6Al-4V titanium alloy.

15. The method of claim 11, wherein analyzing radiation through the initial design comprises analyzing radiation through the initial design via a Monte Carlo simulation.

16. The method of claim 11, wherein analyzing the structural strength of the initial design comprisies analyzing the structural strength of the initial design via finite element analysis.

17. The method of claim 11, wherein the plate comprises a plurality of ribs defining a lattice structure.

18. The method of claim 17, further comprising:
encapsulating the plurality of ribs with collagen.

19. The method of claim 17, further comprising:
inlaying the plurality of ribs with a radiolucent biocompatible membrane.

20. The method of claim 11, wherein the initial design comprises a first amount of material and the altered design comprises a second amount of material, the second amount of material less than the first amount of material.
